(19) United States
(12) Patent Application Publication
(10) Pub. No.: US 2023/0054238 A1
(43) Pub. Date: Feb. 23, 2023

(54) GERMICIDAL DUCT ASSEMBLY

(71) Applicant: Charles E. Rigby, Dawsonville, GA (US)

(72) Inventor: Charles E. Rigby, Dawsonville, GA (US)

(21) Appl. No.: 17/096,193

(22) Filed: Nov. 12, 2020

(51) Int. Cl. F24F 3/16; F24F 13/06; F24F 7/06

(52) U.S. Cl. CPC F24F 3/16; F24F 13/06; F24F 7/065; F24F 2003/1667

(57) ABSTRACT

A germicidal duct assembly that includes an inlet assembly, an irradiation chamber, and an outlet assembly is presented herein. The inlet assembly includes an inlet duct connected at one end to the irradiation chamber and at the other end to a vent. A fan is used to draw air into the inlet assembly and to the irradiation chamber. The outlet assembly includes an outlet duct connected at one end to the irradiation chamber and at the other end to a vent. A fan is used to facilitate the flow of air from the irradiation chamber through the outlet duct. The irradiation chamber includes at least one elongated ultraviolet light source disposed in an oblique manner relative to a longitudinal axis of said irradiation chamber in order to increase the exposure time of the air as it travels through the inlet ducts to irradiation chamber and out of the outlet ducts.

### FIELD OF THE INVENTION

The present invention is generally directed to a disinfection or germicidal duct assembly, and more specifically to a germicidal duct assembly configured to disinfect or sanitize air as it flows through the duct assembly. Other embodiments of the present invention are directed to a system and method for managing, monitoring and controlling a UV light system and UV energy output.

### BACKGROUND OF THE INVENTION

A number of floor and wall-mounted ultraviolet systems are known, but suffer from many defects and drawbacks which lead to ineffective or inefficient disinfecting or germicidal applications of the ambient air in a room. For example, many floor and wall-mounted ultraviolet light systems have an exhaust port that is close in physical proximity to the intake port. This defect creates many untreated zones of air in a room and/or uneven sanitizing of the air in the room.

In addition, some residential heating, ventilating and air conditioning (HVAC) systems may incorporate ultraviolet light intended to disinfect or sanitize the air as it circulates through the HVAC system and the building. One problem with this approach, however, is that a central HVAC system operates to provide heat or air conditioning to an entire building and air is oftentimes exchanged more often and more efficiently in some rooms than in other rooms, for example, depending on the distance the room is from the HVAC system, the size of the room, the number of HVAC vents, etc. Furthermore, since exposure time to a UV or UV-C light source is critical to the effectiveness of disinfection and sanitization, an HVAC system would likely need to cycle air multiple times before a potentially effective sanitization can occur. However, since HVAC systems often do not stay turned on and may only cycle air when a thermostat signals for heating or cooling, these systems have significant inherent flaws, at least for purposes of providing disinfecting and sanitizing capabilities.

Accordingly, there is a need in the art for a more effective and more efficient system that can disinfect and sanitize the air in a single room or multiple rooms of a particular building or structure, whether residential or commercial. The proposed system or assembly can mount to ceilings where other ductwork and electrical lines are located. Inlet and outlet vents can be spaced an optimal distance away from one another (e.g., over twenty feet apart). In addition, the proposed germicidal ducting assembly may be a stand-alone device not connected to an HVAC system, can run or operate continuously regardless of whether a thermostat has triggered the need for heating or cooling, and can be disposed in a single room or multiple rooms, which is not possible for floor and wall-mounted system.

Additionally, the energy output of ultraviolet lamps and light sources inherently degrade over time. For example, current shortwave ultraviolet (UV-C) lamp disinfection systems have a lifetime of 8,000 to 16,000 hours. There are, however, a number of factors in addition to the rated lifetime of the lamp with nominal usages practices that can increase the degradation and therefore decrease the effectiveness of the UV-C light source, including, for example, the number of times the light source is cycled on and off, the type of start process, e.g., preheat start, instant on start, etc. Accordingly, as a UV-C lamp ages and as the lamp is used, its energy levels decrease and eventually the lamp will fail. Efficacy of a lamp to destroy pathogens is directly related to it UV-C energy output. If the irradiance energy of a lamp or light source falls, it becomes less effective at destroying a pathogen thus leaving the pathogens available to infect humans. For these, and potentially other reasons, lamps are routinely replaced at some arbitrary time based on the supposition that they are no longer effective. Worse, in many cases, the lamps are never changed and thus no longer perform as intended or expected.

More in particular, the current effectiveness of a UV-C light source cannot be visually examined since the UV-C radiation will damage the eyes. While there are some manual card-style dosimeters or electronic spectrometers that can be used to measure the energy of a lamp's output, these devices are expensive and must be manually placed and retrieved to obtain a reading. Instead, UV-C lamps are often changed on an estimated maintenance cycle, which may not be accurate. Replacing the lamps too soon can cause unnecessary labor and expenses, while replacing the lamps too late result in an amount of time where the system was not operating effectively.

Accordingly, there is a need in the art for a UV monitoring and managing system and method which extends the maintenance period and removes the guess work from determining the effectiveness of a UV lighting and germicidal system. For instance, the proposed system and method will incorporate sensors, controllers and one or more back-up or reserve UV light sources. When the sensors detect low efficiency or low UV energy, the controller will automatically activate the back-up or reserve UV lights and in some cases can generate a signal indicating that maintenance (e.g., replacing the lamps) is recommended or required.

### SUMMARY OF THE INVENTION

Accordingly, at least one embodiment of the present invention is directed to a germicidal duct assembly that includes an inlet assembly, an irradiation chamber, and an outlet assembly. The germicidal duct assembly can be installed in or on ceilings and operates independent of any HVAC or other system. In other words, the assembly of at least one embodiment can be structured to effectively provide active, localized ventilation and disinfecting/sanitizing in single room or multiple rooms.

More specifically, the inlet assembly may include at least one flexible, semi-rigid or rigid inlet duct or tube connected at one end to the irradiation chamber and at the other end to an inlet vent. A fan can be used to draw air or otherwise facilitate the flow of air into the inlet assembly and to the irradiation chamber where the air will be exposed to a germicidal source, including, but not limited to ultraviolet light emitted by one or more ultraviolet light sources. In some cases, multiple inlet ducts can be connected to a single irradiation chamber, such that air from multiple locations (whether in the same room or different rooms) can be directed into the irradiation chamber. In such a case, each of the inlet ducts can include one or more inlet fans such that air can be drawn into each of the separate inlet ducts.

Similarly, the outlet duct may include at least one flexible, semi-rigid or rigid outlet duct or tube connected at one end to the irradiation chamber and at the other end to an outlet vent. A fan can be used to facilitate the flow of air from the irradiation chamber, through the outlet duct and into the room. In some cases, multiple outlet ducts are connected to a common or single irradiation chamber, such that disinfected or sanitized air can be distributed to multiple locations (whether in the same room or different room). In such a case, each of the outlet ducts or outlet assemblies can include one or more fans such that air can be directed from the irradiation chamber through each of the outlet ducts.

Furthermore, the irradiation chamber of the at least one embodiment of the present invention comprises at least one ultraviolet light source disposed within an interior portion thereof. Instead of, or in addition to the at least one ultraviolet light source, some embodiments may include a bipolar ionization source or generator, a photocatalytic oxidation (PCO) source or generator, or other germicidal sources or air purification systems. In some embodiments, the ultraviolet light source(s) or other germicidal sources have an elongated configuration and are disposed in an oblique manner relative to a longitudinal axis of said irradiation chamber. In the case of ultraviolet light sources, this ensures that some UV light shines into or is emitted into the inlet assembly, e.g., into the inlet duct(s) and/or an optional connecting pipe, and/or into the outlet assembly. This increases the exposure time of the air as it travels through the inlet ducts to irradiation chamber and out of the outlet ducts.

In some embodiments, a reflecting material or surface is provided on any one or more of the interior of the inlet duct(s), the interior of a connecting pipe, the interior of the irradiation chamber, and/or the interior of the outlet duct. For instance, in at least one embodiment, the reflective material or surface may include a diffusely reflective surface, thereby causing Lambertian reflectance within the assembly. The reflective material or surface acts similar to a photomultiplier or diffuser to direct the iridescence energy more evenly and widely throughout the pipe(s), duct(s) or tube(s), and may in some instances increase the iridescence energy in the pipe(s), duct(s) or tube(s). As just an example, the reflecting material or surface of at least one embodiment may be constructed of or otherwise include polytetrafluoroethylene (PTFE), although other reflective or diffusing materials are contemplated.

Furthermore, in other embodiments, the present invention is directed to or otherwise includes one or more sensors, including but not limited to a UV sensor, that can measure the energy level of UV light, for example, in the irradiation chamber or in another UV light or germicidal system. The sensors can be connected to a controller or circuitry that compares the measured energy level with a predetermined lower threshold amount. The predetermined lower or threshold amount may be defined by a user or preset by the manufacturer or operator. When the measured UV energy reaches or falls below the predetermined level, backup or reserve UV light sources can be automatically activated or turned on in order to increase the UV energy level. For example, the active UV light sources can either remain on (and therefore continue to produce some UV light) or be disconnected when the backup or reserve lights are turned on or activated. The sensors and system can then continue to monitor the UV energy output level to determine if or when additional maintenance (e.g., replacing the light sources or lamps) is needed or if available, additional backup or reserve lights are activated.

In addition, one or more light emitting diode(s), LED(s), indicator lamp(s) or display(s) can be installed or mounted on the system or in a separate remote location to indicate the current UV energy level, when maintenance is required or recommended, or the general status of the system.

In this manner, the system and method of at least one embodiment can provide for continuous, effective disinfection of pathogens by maintaining desired UV energy levels. It also extends the need to replace lamps by having an automated backup lamp system that can provide for visual or audible indication that the lamps are nearing the end of their life cycle.

These and other objects, features and advantages of the present invention will become more apparent when the drawings as well as the detailed description are taken into consideration.

### BRIEF DESCRIPTION OF THE DRAWINGS

FIG. 1A is an elevation view of the germicidal duct assembly as disclosed in accordance with at least one embodiment of the present invention.

FIG. 1B is an exploded view of the germicidal duct assembly illustrated in FIG. 1A.

FIG. 2A. is an elevation view of the germicidal duct assembly as disclosed in accordance with another embodiment of the present invention.

FIG. 2B is an exploded view of the germicidal duct assembly illustrated in FIG. 2A.

FIG. 3A is an exploded perspective view of the inlet vent, inlet fan and a portion of the inlet duct as disclosed in accordance with another embodiment of the present invention.

FIG. 3B is another exploded perspective view of the inlet vent, inlet fan and a portion of the inlet duct as disclosed in accordance with another embodiment of the present invention.

FIG. 4A is a perspective view of the fan mount as disclosed in accordance with at least one embodiment of the present invention.

FIG. 4B is another perspective view of the fan mount illustrated in FIG. 4A.

FIG. 4C is an elevation view of the fan mount illustrated in FIGS. 4A and 4B.

FIG. 5 is a perspective exploded view of the outlet assembly and coupler as disclosed in accordance with at least one embodiment of the present invention.

FIG. 6 is a cut-away, perspective view of the irradiation chamber as disclosed in accordance with at least one embodiment of the present invention.

FIG. 7A is a perspective view from the second end of the irradiation chamber with one UV light source, as disclosed in accordance with at least one embodiment of the present invention.

FIG. 7B is a perspective view from the first end of the irradiation chamber shown in FIG. 7A.

FIG. 7C is an end view of the irradiation chamber with one UV light source as disclosed in accordance with at least one embodiment of the present invention.

FIG. 8 is an end view of the irradiation chamber of another embodiment with three UV light sources.

FIG. 9A is a perspective view of the irradiation chamber of another embodiment with a plurality of UV light sources.

FIG. 9B is a cut-away view of the irradiation chamber of at least one embodiment with four UV light sources.

FIG. 9C is a side and partial cut-away view of the irradiation chamber illustrated in FIG. 9B.

FIG. 9D is an end view of the irradiation chamber illustrated in FIGS. 9B and 9C.

FIG. 10A is a perspective view of the irradiation chamber of yet another embodiment of the present invention.

FIG. 10B is a cut away view of the irradiation chamber illustrated in FIG. 10A.

FIG. 10C is an end view of the irradiation chamber illustrated in FIGS. 10A and 10B.

FIG. 11A is a partial cut-away view of the connecting pipe illustrating a reflective surface or material disposed therein in accordance with at least one embodiment of the present invention.

FIG. 11B is a partial cut-away view of the inlet duct illustrating a reflective surface or material disposed therein in accordance with at least one embodiment of the present invention.

FIG. 11C is a partial cut-away view of the irradiation chamber illustrating a reflective surface or material disposed therein in accordance with at least one embodiment of the present invention.

FIG. 11D is a partial cut-away view of the outlet duct illustrating a reflective surface or material disposed therein in accordance with at least one embodiment of the present invention

FIG. 12A is an end view of the irradiation chamber of at least one embodiment of the present invention illustrating one UV light source, a sensor and a controller.

FIG. 12B is a perspective view of the irradiation chamber of at least one embodiment of the present invention illustrating a plurality of UV light sources, at least one sensor and at least one controller.

FIG. 13 is a high level flow chart illustrating the method of managing, monitoring and controlling a UV light system as disclosed in accordance with at least one embodiment of the present invention.

FIG. 14A is an exemplary view of a human-machine interface for the system and method for managing, monitoring and controlling a UV light system and UV energy output as disclosed in accordance with at least one embodiment of the present invention.

FIG. 14B is another exemplary view of a human-machine interface for the system and method for managing, monitoring and controlling a UV light system and UV energy output as disclosed in accordance with at least one embodiment of the present invention.

FIG. 14C is another exemplary view of a human-machine interface for the system and method for managing, monitoring and controlling a UV light system and UV energy output as disclosed in accordance with at least one embodiment of the present invention.

FIG. 14D is yet another exemplary view of a human-machine interface for the system and method for managing, monitoring and controlling a UV light system and UV energy output as disclosed in accordance with at least one embodiment of the present invention.

Like reference numerals refer to like parts throughout the several views of the drawings provided herein.

### DETAILED DESCRIPTION OF THE INVENTION

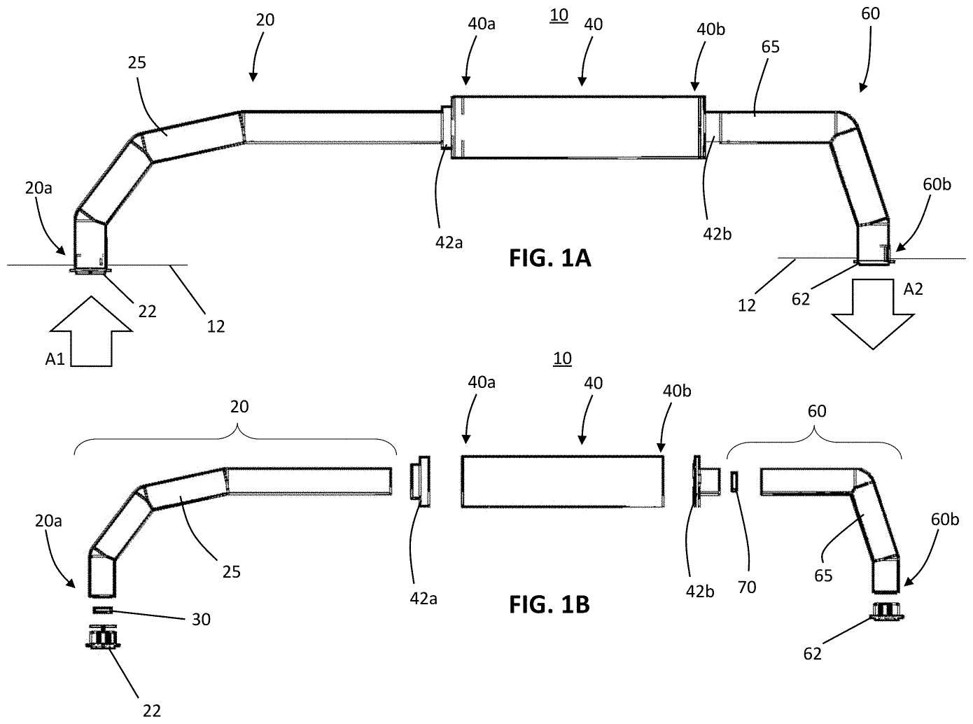

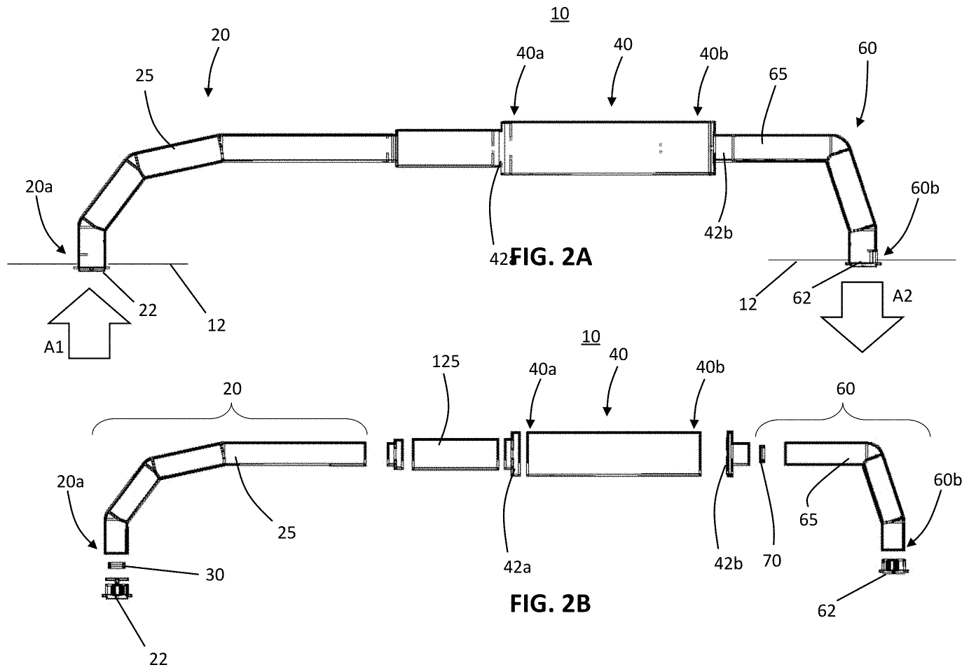

As shown in the accompanying drawings, and with particular reference to FIGS. 1A and 2A, the present invention is generally directed to a disinfecting or germicidal duct assembly referenced as 10. Specifically, the duct assembly 10 of certain embodiments of the present invention is a stand-alone, independent or local system that uses a germicidal source, such as, for example ultraviolet (UV) light, including but not limited to short-wave ultraviolet light (often referred to as UV-C light) or light having a wavelength in the range of approximately 100 nm to 280 nm, to disinfect or sanitize air as the air flows through the assembly 10 and is exposed to the UV light. In other words, the assembly 10 of at least one embodiment is not part of or connected to a standard heating, ventilation and air conditioning (HVAC) system, but rather operates, creates a flow of air and disinfects on its own and independent of any HVAC or other external systems. It should be noted that other germicidal or disinfecting sources can be used in connection with the various embodiments disclosed herein instead of or in addition to one or more UV light sources. For example, the germicidal or disinfecting source(s) may include bipolar ionization sources, generators or technology. More in particular, bipolar ionization sources put positive and negative ions into the air that can then be distributed into one or more rooms in connection with the duct assembly of the present invention. The ions can then kill or inactivate bacteria, viruses, mold, volatile organic compounds (VOC), odors, and cause minute particles in the air to coalesce into larger particles that can be caught by an air filter. Furthermore, other germicidal or disinfecting sources that can be used may include photocatalytic oxidation (PCO) sources or generators, for example.

Furthermore, the duct assembly 10 of at least one embodiment of the present invention is intended to be installed or mounted overhead, for example, above or within a drop ceiling, as generally referenced as 12 in FIGS. 1A and 2A (e.g., in a classroom, office, senior care home, etc.) or on or in open ceilings (e.g., often found in retail stores, restaurants, warehouses, manufacturing plants, etc.).

Still referring to FIGS. 1A and 2A, for example, the disinfecting duct assembly 10 of at least one embodiment includes an inlet assembly 20, an irradiation chamber 40, and an outlet assembly 60. As will become apparent from the disclosure provided herein, air (for example, from a room in a building, home or other structure) flows or is drawn into the inlet assembly 20, as shown by reference arrow A1, through the irradiation chamber 40, and out of the outlet assembly 60, as shown by reference arrow A2 (for example, back into the same room or into a different room.)

One or more disinfecting or germicidal sources, such as, for example, a germicidal light sources is/are disposed within the irradiation chamber 40, such that, as the air flows through the irradiation chamber 40, the air is exposed to the disinfecting or germicidal source, such as ultraviolet light emitted by the light source(s), which in turn will disinfect or sanitize the air. The disinfected or sanitized air will then flow through the outlet assembly 60 and back into the room or into a different room.

In this manner, it should be noted that the disinfecting duct assembly 10 of at least one embodiment may be disposed in a single room (e.g., such that air from the room will flow into the assembly 10 and back into the same room) or span across multiple rooms (such that air from one or more rooms will flow into the assembly 10 and out into one or more different rooms).

In any event, referring to FIGS. 1A through 2B, the irradiation chamber 40 of at least one embodiment includes a first end 40a connected to the inlet assembly 20 and a second end 40b connected to the outlet assembly 60. The inlet assembly 20, irradiation chamber 40 and outlet assembly 60 collectively define an interior pathway through which the air is able to flow. As just an example, the irradiation chamber 40 may be constructed of an elongated, cylindrical pipe or rectangular duct, such as, but not limited to a galvanized steel round pipe or rectangular duct. In some embodiments, the irradiation chamber 40 may be twenty-four to sixty inches in length and have a twelve inch diameter, although other shapes, sizes and materials are contemplated within the full spirit and scope of the present invention.

Moreover, the inlet assembly 20 of at least one embodiment includes one or more inlet ducts 25 constructed out of or otherwise including one or more flexible, semi-rigid or rigid ducts, pipes, or tubes (e.g., a standard HVAC duct) and can include an inlet vent 22 or opening at an inlet end 20a thereof. As just an example, the inlet duct(s) 25 may be a six inch diameter flexible round duct, although other sizes, shapes and materials are contemplated. For instance, the inlet duct(s) 25 may be flexible and therefore easily positionable or movable, while in other embodiments, the inlet duct(s) 25 may be rigid or semi-rigid.

In some cases, a coupler, such as an end cap, reducer or increaser can be used or disposed at the first end 40a of the irradiation chamber 40 to facilitate connection between the inlet assembly 20 and the irradiation chamber 40. For example, if the connecting diameter of the inlet assembly 20 is smaller than the connecting diameter of the irradiation chamber 40, a coupler 42a or increaser may be needed such that one end of the coupler 42a is sized and configured to connect to the inlet assembly 20 and the other end of the coupler 42a is sized and configured to connect to the irradiation chamber 40.

It should also be noted that, although not shown in the drawings, the inlet assembly 20 can include a plurality of inlet ducts 25 each having separate inlet vents 22 and each connected to or independently communicative with the irradiation chamber 40. For example, the single end of a “Y” shaped connector (not shown) can connect to the first end 40a of the irradiation chamber 40 such that two (or more) separate inlet ducts 25 can connect to the irradiation chamber 40, thereby allowing air from two (or more) separate locations (either in the same room or different rooms) to enter into the same irradiation chamber 40.

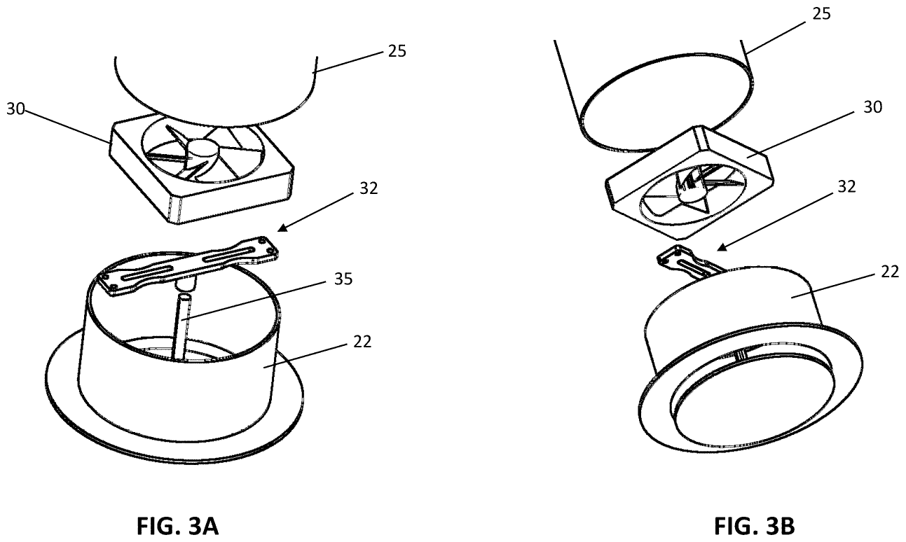

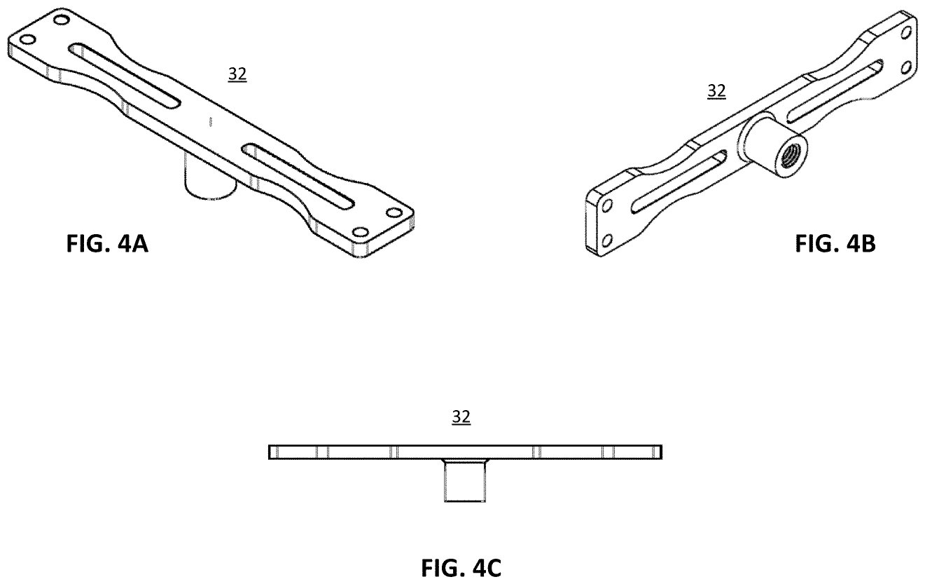

In addition, with reference to FIGS. 1B, 2B, and 3A through 4C, for example, the inlet assembly 20 of at least one embodiment includes an inlet fan 30 structured and configured to draw air into or otherwise facilitate the flow of air into the inlet assembly 20. As an example, the inlet fan 30 may be mounted to the inlet vent 22 with a mounting bracket 32. In this manner, air will be drawn through the opening(s) 23 of vent 22 by the fan 30 and pass or flow through the inlet assembly 20 to the irradiation chamber 40. The mounting bracket 32, for example, may attach to a rod 35 which extends from the vent 22. Mounting holes or slots in the bracket can then be used to mount the fan 30 thereto. It should be noted, however, that the inlet fan 30 can be connected to the assembly 10 along virtually any portion of the inlet assembly 20 for facilitating the flow of air into the inlet assembly 20 as provided in accordance with a number of embodiments disclosed herein.

Some embodiments of the inlet assembly 20, however, may not need or have an inlet fan 30. For example, as disclosed below, the outlet assembly 60 of some embodiments may include one or more outlet fans 70. In some cases, the outlet fan(s) 70 may be large enough or powerful enough to draw air into the inlet assembly 20 such that additional inlet fan(s) 30 may not be necessary.

Further, the outlet assembly 60 of at least one embodiment includes one or more outlet ducts 65 constructed out of or otherwise including one or more flexible or rigid ducts, pipes, or tubes (e.g., a standard HVAC duct) and can include an outlet vent 62 or opening at an outlet end 60B. As just an example, the outlet duct(s) 65 may be a six inch diameter flexible round duct, although other sizes, shapes and materials are contemplated. For instance, similar to the inlet duct(s) 25, the outlet duct(s) 65 of some embodiments may be flexible and therefore easily positionable or movable, while in other embodiments, the outlet duct(s) 65 may be rigid or semi-rigid.

Further, in some cases, a coupler, such as an end cap, reducer or increaser can be used or disposed at the second end 40b of the irradiation chamber 40 to facilitate connection between the outlet assembly 60 and the irradiation chamber 40. For example, if the connecting diameter of the outlet assembly 60 is smaller than the connecting diameter of the irradiation chamber 40, a coupler 42b or reducer may be needed such that one end of the coupler 42b is sized and configured to connect to the irradiation chamber 40 and the other end of the coupler 42b is sized and configured to connect to the outlet assembly 60.

It should also be noted that, although not shown in the drawings, the outlet assembly 60 can include a plurality of outlet ducts 65 each having separate outlet vents 62 and each connected to or independently communicative with the irradiation chamber 40. For example, the single end of a “Y” shaped connector, not shown, can connect to the second end 40b of the irradiation chamber 40 such that two (or more) separate outlet ducts 65 can connect to the irradiation chamber 40, thereby allowing disinfected air to flow out of the irradiation chamber 40 and into two or more separate locations (either in the same room or different rooms).

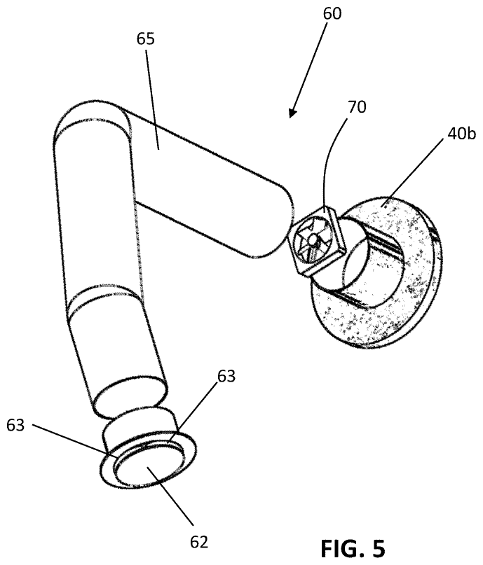

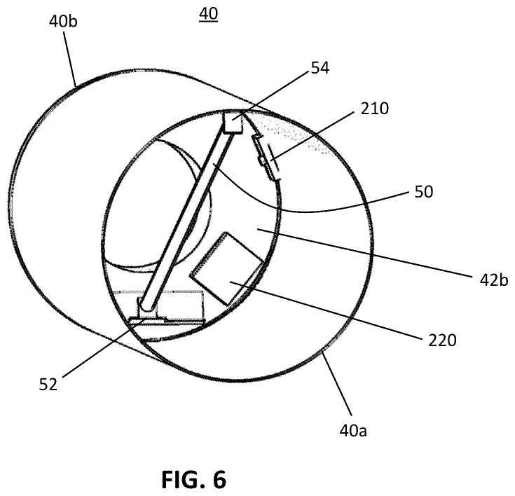

In addition, with reference to FIGS. 1B, 2B, and 5, for example, the outlet assembly 60 of at least one embodiment includes an outlet fan 70 structured and configured to facilitate the flow of air out of the irradiation chamber 40 and/or out of the outlet assembly 60. As an example, the outlet fan 70 may be mounted proximate the second end 40b of the irradiation chamber, for example, to or within coupler 40b which extends from or is otherwise attached to the irradiation chamber 40. In this manner, disinfected air will flow out of the irradiation chamber 40 via the fan 70 and through the opening(s) 63 of vent 62.

Some embodiments of the outlet assembly 60, however, may not need or have an outlet fan 70. For example, as disclosed above, the inlet assembly 20 of some embodiments may include one or more inlet fans 30. In some cases, the inlet fan(s) 30 may be large enough or powerful enough to draw air into the inlet assembly 20 and direct the air through the irradiation chamber 40 and out of the outlet assembly 60 such that additional outlet fan(s) 70 may not be necessary.

In this manner, the germicidal duct assembly 10 of at least one embodiment includes at least one fan 30, 70 disposed in or proximate the inlet assembly 20 (e.g., an inlet fan 30) and/or disposed in or proximate the outlet assembly 60 (e.g., an outlet fan 70).

In any event, with reference now to the exploded view of FIG. 6, the irradiation chamber 40 of at least one embodiment includes at least one disinfecting or germicidal source, such as a disinfecting or germicidal light source 50 disposed therein. In particular, the disinfecting or germicidal light source 50 of at least one embodiment is a light bulb, light tube, light emitting diode (LED) light or other structure that emits germicidal ultraviolet light. More specifically, in at least one exemplary embodiment, the disinfecting or germicidal light source 50 is structured to emit short wavelength ultraviolet light (UV-C light) or light having a wavelength in the range of approximately 100 nm to 280 nm, although light having other wavelengths may be used.

For instance, the disinfecting or germicidal light source 50 of at least one embodiment may be mounted to an inside surface of one of the couplers 42a, 42b and extend inward through at least a portion of the irradiation chamber 40. As an example, one or more mounting holes may be provided in coupler 42b, to which a light socket 52 can be mounted. The light source or bulb 50 can then connect or mount to the socket 52 and extend within the irradiation chamber 40. A support mount 54 may be secured to the opposing end of the light source 50, as shown in FIG. 6, for example. The support mount 54 may be attached or mounted to an inside surface of the irradiation chamber 40, to the first coupler 42a or other location spaced a distance from the socket 52.

In some instances, the light source(s) 50 include(s) an elongated configuration (e.g., as shown in FIG. 6, and can extend at least substantially along the length of the irradiation chamber 40. More specifically, the light source(s) 50 may begin at or near one coupler 42b and extend into and through the irradiation chamber 40 to the other coupler 42a or proximate the other coupler 42a, for example, at first end 40a of the irradiation chamber 40. In particular, in some cases, as shown in FIG. 6, the end or support mount 54 may connect to the inside surface of the irradiation chamber 40 proximate the first end 40a thereof.

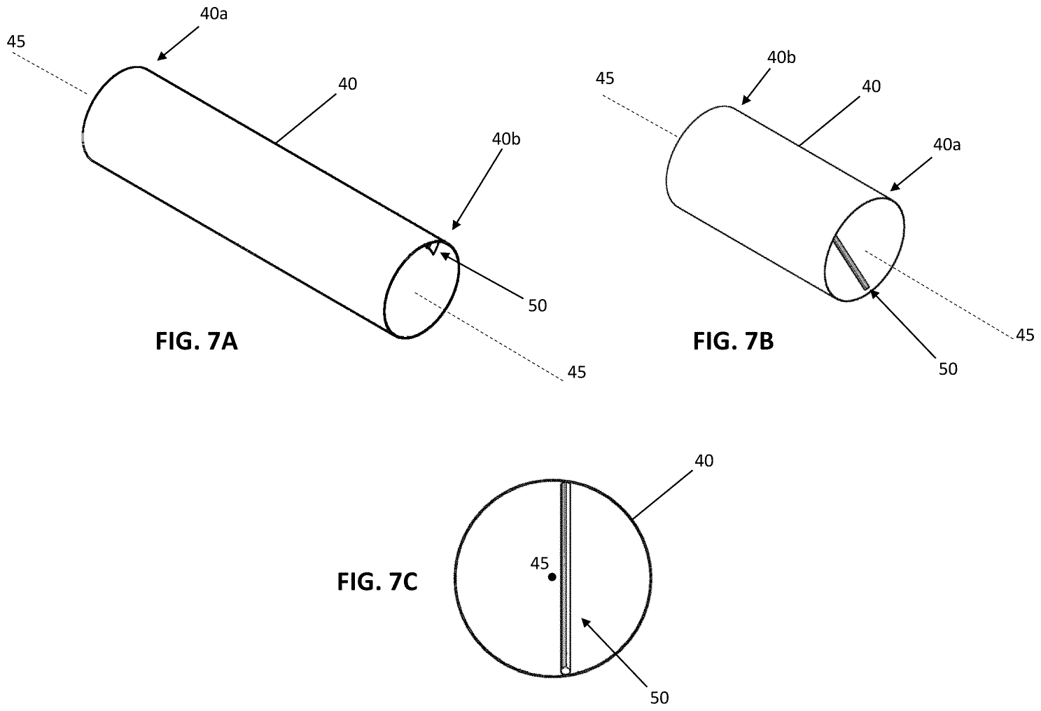

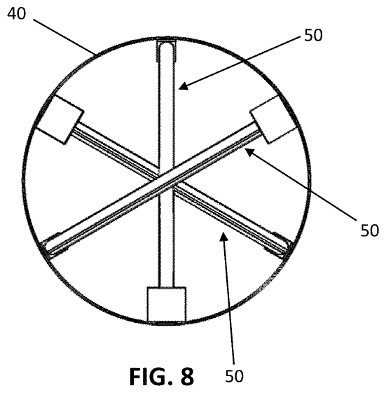

In addition, the one or more light sources 50 of at least one embodiment is/are disposed in an oblique, staggered or angled manner relative to a longitudinal axis 45 of the irradiation chamber 40. More specifically, in at least one embodiment, the irradiation chamber 40 comprises a tube-like configuration defined as a cylinder with opposing ends, such as first end 40a and second end 40b. With reference to FIGS. 7A and 7B, a longitudinal axis 45 is defined as extending longitudinally through the center of the chamber 40, as illustrated.

More specifically, the oblique disposition of the one or more light sources 50 is such that the elongated light sources is/are not parallel to the longitudinal axis 45 of the irradiation chamber 40. This angular, staggered or oblique positioning of the light source(s) 50 allows the emitted germicidal ultraviolet light to shine or travel at least partially into the inlet assembly 20. By doing so, the air that travels or flows through the assembly 10 of at least one embodiment of the present invention may be exposed to the germicidal ultraviolet light prior to entering or flowing into the irradiation chamber 40, as well as while the air is in the irradiation chamber 40. This provides additional exposure time to the air that flows through the assembly 10 of certain embodiments of the present invention.

For example, with reference to FIGS. 7A, 7B and 7C, the irradiation chamber 40 of at least one embodiment is shown along with the longitudinal axis 45 and a single light source 50 disposed therein. As provided below, additional light source(s) 50 can be included to give more disinfecting or germicidal power. For example, in the embodiment that has multiple inlet ducts 25 and multiple inlet vents (e.g., coming from the same or different rooms), UV energy or power may need to be increased by increasing the number of light sources 50 disposed in the irradiation chamber 40.

As represented in FIGS. 7B and 7C, the light source 50 spans through the irradiation chamber 40 and crosses from one internal side of the chamber 40 to the other, and is therefore disposed in an oblique manner and not parallel to the axis 45. More specifically, FIG. 7A shows and end perspective view from second end 40a of the irradiation chamber 40 with the light source 50 connected to a top or upper end 41a of the irradiation chamber 40. FIG. 7B shows the same irradiation chamber 40 in a perspective view from the first end 40a illustrating the light source is connected to or extends to an opposite or lower side 41b of the irradiation chamber 40.

Similarly, FIG. 8 shows an end view of the irradiation chamber 40 of at least one embodiment with three light sources 50 disposed therein. As shown, the three light sources 50 are disposed in an oblique or angled manner relative to each other and relative to the longitudinal axis 45. In this embodiment, each of the three light sources 50 also extend through the longitudinal axis 45 such that in the end view (as shown in FIG. 45) the light sources 50 are symmetrically disposed, although the light sources 50 do not collide or contact one another.

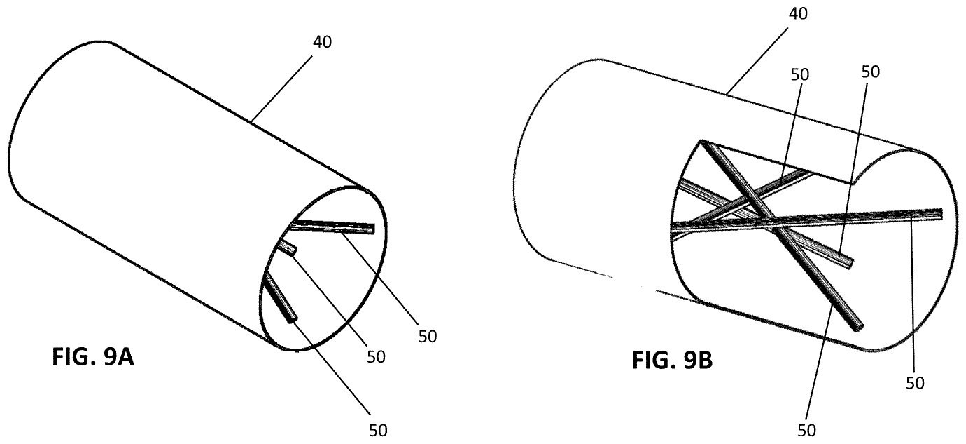

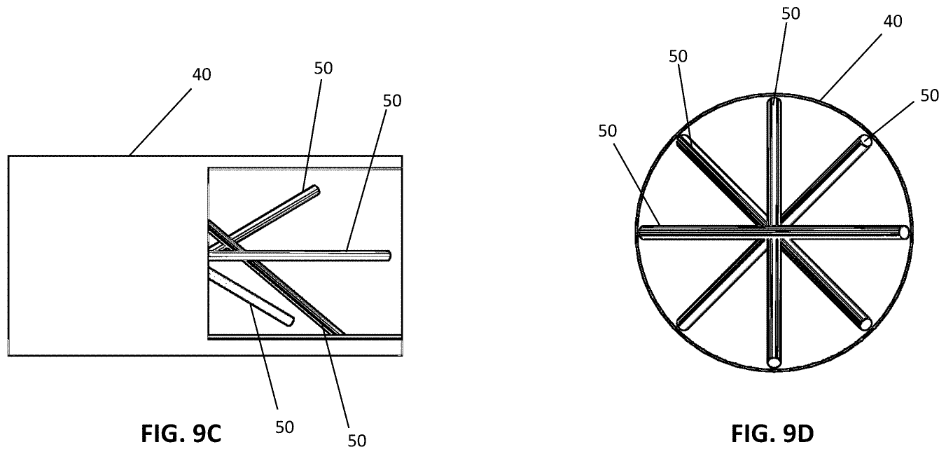

FIGS. 9A through 9D illustrate another embodiment of the irradiation chamber 40, this time with four light sources 50 disposed in an oblique, staggered or angled manner therein. More specifically, FIGS. 9B and 9C illustrate partial cut-away views of the irradiation chamber 40 showing how the various light sources 50 are angularly disposed or obliquely disposed relative to one another and relative to a longitudinal axis 45 without the light sources 50 colliding with one another. When viewed from the end, such as in FIG. 9D, the light sources 50 appear symmetrically positioned.

For instance, the oblique or staggered light source arrangement allows the lights to be staggered in the chamber 40 to achieve a symmetrical axis view without the light sources 50 colliding in the middle. This can be scalable by increasing the chamber length to hold more light sources and to add to the axial light density. The chamber diameter can also be increased thereby increasing the light tube angle therein.

In addition, it should also be noted that with the obliquely or angularly positioned light source(s) 50, as the air flows through the irradiation chamber 40, the flowing air will impact the light source(s) 50 and cause the air to break up or mix, thereby increasing exposure and/or exposure time to the ultraviolet light as the air passes from the inlet assembly 20 to the outlet assembly 60.

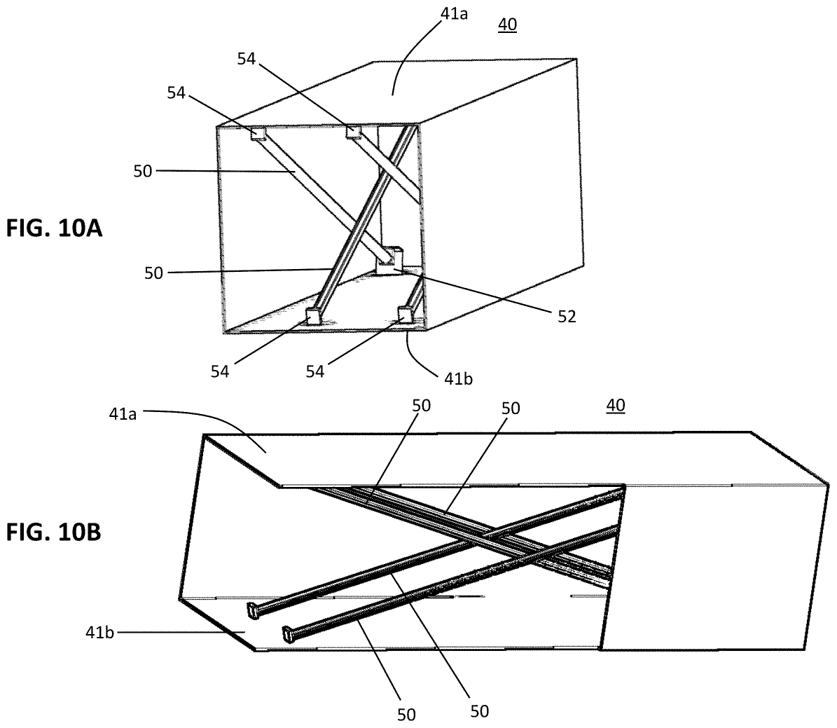

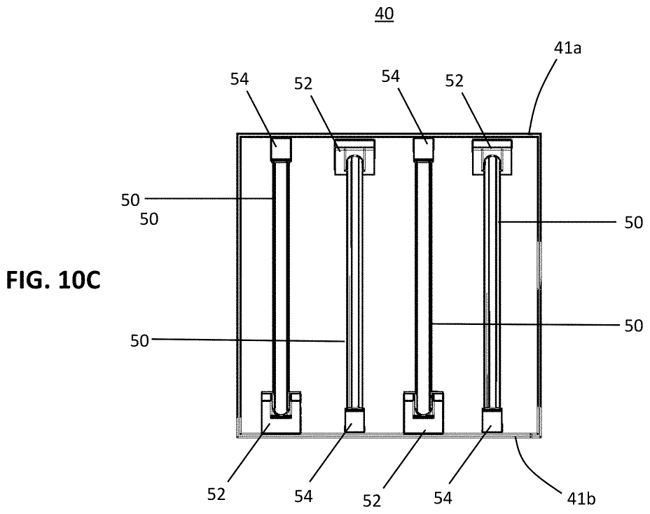

Furthermore, FIGS. 10A, 10B and 10C illustrate yet another embodiment of the irradiation chamber 40 of the present invention, showing a rectangular irradiation chamber 40, or otherwise, an irradiation chamber 40 with a rectangular cross-section. In this embodiment, the plurality of light sources 50 are shown as being disposed in an oblique orientation relative to the longitudinal axis 45 of the chamber 40. In this example, each light source 50 spans from one end (e.g., a top 41a or bottom end) of the chamber 40 to the other opposite end of the chamber 40. In other implementations, the light sources 50 may be arranged from side-to-side, or in another oblique or staggered arrangement.

Referring again to FIGS. 2A and 2B, it should also be noted that the inlet assembly 20 of at least one embodiment of the present invention may include an optional connection pipe or duct 125 disposed between the irradiation chamber 40 and the flexible inlet duct(s) 25. The connection pipe or duct 125 may be constructed of an elongated, cylindrical pipe or rectangular duct, such as, but not limited to a galvanized steel round duct pipe or rectangular duct. In some embodiments, the connection pipe 125 may be twenty to forty eight inches in length and have an eight or ten inch diameter, although other shapes, sizes and materials are contemplated. In the event the connection pipe 125 has a diameter that is different than that of the flexible duct 25 and/or irradiation chamber 40, couplers, such as increasers or reducers may be needed, as described above, to facilitate interconnection there between.

The optional connection pipe or ducts 125 can be used as a pre-exposure chamber such that the germicidal light from the light source(s) 50 of the irradiation chamber 40 may shine or travel into the connection pipe or duct 125, thereby exposing the air to the germicidal UV light prior to the air reaching to or travelling through the irradiation chamber 40.

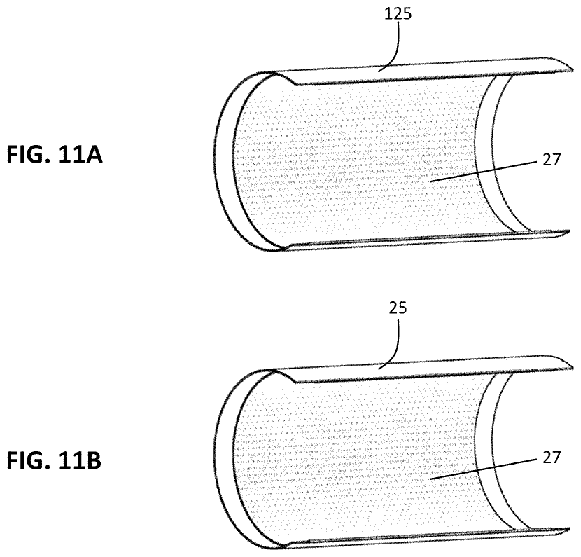

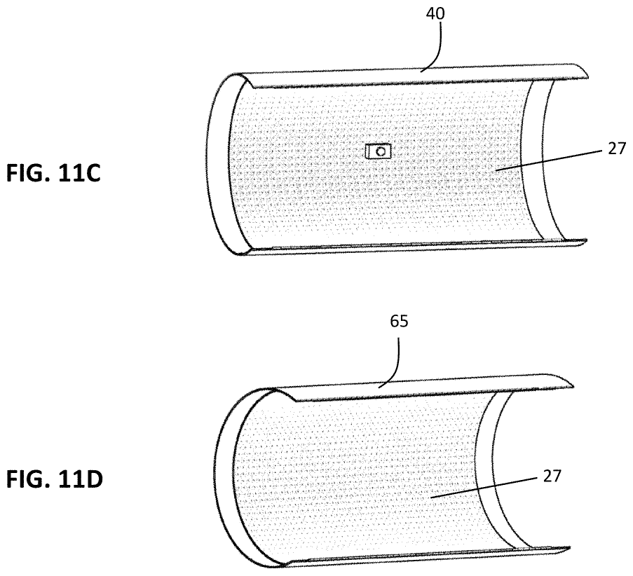

Moreover, in at least one embodiment, with reference to FIG. 11A, at least some or all of the interior surface of the connection pipe 125 may include a light reflecting material or surface 27 disposed thereon. FIGS. 11B, 11C and 11D are provided to also illustrate that, in some embodiment, the light reflecting material or surface 27 may be disposed on all of some of the interior surfaces of the inlet duct(s) 25 (e.g., FIG. 11B), the irradiation chamber 40 (e.g., FIG. 11C) and/or the outlet duct(s) 65 (e.g., FIG. 11D). More specifically, the light reflecting material or surface 27 may be included on the interior surface of any one of the connection duct/pipe 125, inlet duct(s) 25, irradiation chamber 40, or outlet duct(s) 65, each one of the connection duct/pipe 125, inlet duct(s) 25, irradiation chamber 40, and outlet duct(s) 65, or a combination of two or more of the connection duct/pipe 125, inlet duct(s) 25, irradiation chamber 40, and/or outlet duct(s) 65.

More in particular, the light reflecting material or surface 125 of at least one embodiment is structured to be highly reflective and capable of facilitating the germicidal or disinfecting light emitted from the light source(s) 50 to reflect and travel through the inlet duct(s) 25, the connecting pipe or duct 125, the irradiation chamber 40 and/or the outlet duct(s) 65. In other words, the reflective material or surface 27 may, in some embodiments be disposed on a portion of or the entire inside surface of the connection duct/pipe 125, inlet duct(s) 25, irradiation chamber 40, and/or outlet duct(s) 65.

Specifically, in at least one embodiment, the reflective material or surface 27 may include a diffusely reflective surface, thereby causing Lambertian reflectance which is the property that defines an ideal matte or diffusely reflective surface and is named after Johann Heinrich Lambert who introduced the concept of perfect diffusion. In other words, the reflective material or surface 27 acts similar to a photomultiplier to direct the iridescence energy more evenly and widely throughout the pipe(s), duct(s) or tube(s), and may in some instances increase the iridescence energy in the pipe(s), duct(s) or tube(s). In some cases, the Lambertian reflectance of the interior surface(s) of either one or more of the connection duct/pipe 125, inlet duct(s) 25, irradiation chamber 40, and/or outlet duct(s) 65 can be near 90% to 97%. As just an example, the reflecting material or surface may be constructed of or otherwise include polytetrafluoroethylene (PTFE), although other reflective or diffusing materials are contemplated. For instance, one such reflective material or surface is the POREX® VIRTEK® PTFE material provided by POREX FILTRATION GROUP®.

Furthermore, referring back to FIG. 6, in some embodiments mounting holes (not shown) may be formed within the coupler 42b in order to mount the lamp socket(s) 52, an electronic ballast, and a fan power supply. Additional holes 43 can be used to route an AC electrical connection (not shown) to provide power to the light source(s), fan(s) and any one or more sensor(s) described below. In some cases, the inlet fan 30 can be powered by a power line running through the irradiation chamber 40 and down the inside of the inlet duct(s) 25. Other manners of powering the light source(s) 50, fan(s), sensor(s), etc. are contemplated within the full spirit and scope of the present invention.

Yet another embodiment of the present invention includes one or more ultraviolet or UV sensors 210 and a controller 220 to automatically or in response to human input control the light output of one or more of the light source(s). The controller 220 may include various control logic, electrical relays and other components and circuits that are structured and configured to operate the system or method of at least one embodiment as described below. In some embodiments, a human-machine interface (HMI) may be included to provide information (e.g., as determined by the one or more sensors) to a user and to provide controls to the user to enter data, upper or lower energy limits, etc.

It should also be noted that the UV sensor(s) 210, controller(s) 220 and/or interface described herein can be implemented and operable in connection with the germicidal duct assembly 10, while in other cases, the UV sensors, controller(s) and/or interface can be implemented independent of the germicidal duct assembly 10. In other words, the present application discloses a UV controlling system and method, using the UV sensor(s) and controller(s) described herein, which may be implemented with the presently described germicidal duct assembly 10 or with other UV systems (separate and apart from the germicidal duct assembly 10) now known or later developed.

In any event, with reference to FIGS. 12A, 11B and 13 an exemplary system 200 and method 300 are shown. The system 200 of at least one embodiment includes at least one disinfecting or germicidal light source 250 (e.g., as shown in FIG. 11A) or a plurality of disinfecting or germicidal light sources 250a, 250b, 250c 250d, which is/are structured to emit germicidal or disinfecting light such as ultraviolet (UV) light, and in some instance, short-wave ultraviolet light (often referred to as UV-C light) or light having a wavelength in the range of approximately 100 nm to 280 nm. These lights 250, 250a-d and system 200 can be used in connection with the germicidal duct assembly 10 disclosed herein such that the lights 250, 250a-d extend into an irradiation chamber 40 where a flow of air is exposed to the lights. In other cases, the system 200 and lights 250, 250a-d can be used with virtually any germicidal or disinfecting product configured to disinfect air or items exposed to the light.

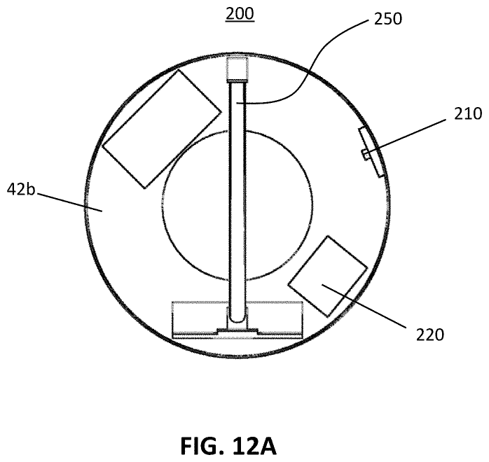

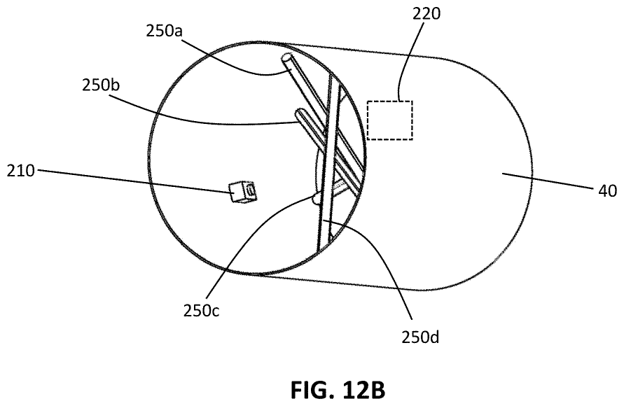

Furthermore, and still referring to FIGS. 12A and 12B, the system 200 also includes one or more sensors, referenced as 210, and one or more controllers, referenced as 220. The sensor(s) 210 can be any ultraviolet light sensors or shortwave ultraviolet light (UV-C) sensors that are exposed to the light emitted by the one or more light sources 250, 250a-d. The sensor(s) 210 can be communicatively connected to the controller 220 as well as a power supply (not shown). The controller 220 may be mounted virtually anywhere in the system 200, for example, on or within the chamber 40, coupler 42b, internally or externally to the system, locally or remotely to the system 220, etc.

Moreover, the sensor(s) 210 are structured and configured to measure or monitor the energy level of the UV light(s) within the chamber or of the UV lighting product or system. Briefly, with multiple UV light sources included in the system (e.g., as shown in FIG. 12B) when the energy level measured by the sensor(s) reaches or falls below a predetermined amount, the system 200 and method 300 will activate an inactive UV light source 250a-d to raise the UV energy level output.

More specifically, the system 200 of at least one embodiment includes at least one active germicidal or UV light source (e.g., 250a) and at least one back-up or reserve germicidal or UV light source (e.g., 250b, 250c, 250d). The back-up or reserve germicidal or UV light source(s) 250b, 250c, 250d are at least initially inactive or otherwise turned off and not emitting UV light. It is contemplated that, in some cases, the back-up or reserve germicidal or UV light source(s) can be configured to emit a small amount of light when in the deactivated state.

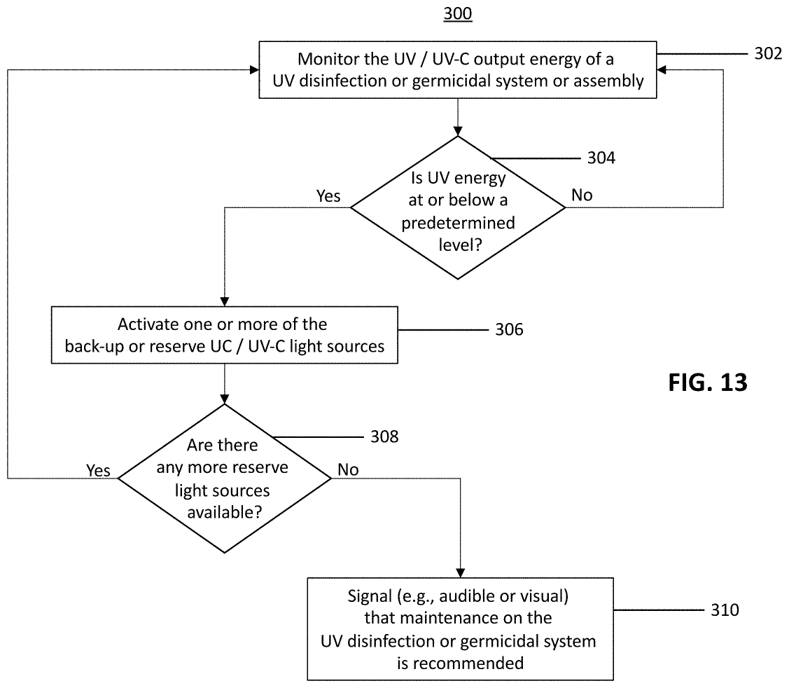

Accordingly, as represented at block 302 in FIG. 13, the sensor(s) 210 are configured to monitor the output energy or UV energy of a UV disinfecting or germicidal system. In many cases, less than all of the UV lights are active (e.g., 250a) at one time, leaving the other lights (e.g., 250b, 250c, 250d) off or inactive. With reference to 304 in FIG. 13, the controller 220 or control logic therein will determine, based on the information or data provided by the sensor(s) 210, whether the energy level emitted by the UV lights is at or below a predetermine threshold. If it is not, then the sensor(s) 210 continue to monitor the energy level with no change.

If, on the other hand, the energy level reaches or falls below a predetermined level (e.g., due to ballast failure, lamp degradation over time, lamp degradation due to debris build-up, etc.), then, as shown at 306, the controller 220 of at least one embodiment will activate one or more of the back-up or reserve UV lights (e.g., 250b, 250c, 250d). In that case, the back-up or reserve UV lights will then be converted to active UV lights thereby emitting a high UV energy level. In some cases, the previous active light (250a) may be completely deactivated or turned off, while in other cases, it may be left to continue to emit UV energy, if any.

For example, in some cases, when the energy level measured by the sensor(s) 210 is too low or otherwise reaches a predetermine lower limit, the system or controller 220 may activate an electrical relay or circuit path that will power one or more of the back-up or reserve UV light(s) 250b, 250c, 250d. Other manners of activating one or more of the back-up or reserve UV lights is contemplated. In any case, with the back-up or reserve UV lights activated, the energy level in the chamber or otherwise emitted by the system 200 will increase to an operable level.

The cycle then repeats itself until the last reserve UV light(s) in the irradiation chamber or system have reached or fallen below the lower threshold level. For instance, with reference to 308 in FIG. 13, if, after activating one or more reserve UV lights, there are still some remaining reserve UV lights that have not been activated, then the process or method 300 will continue to monitor the UV energy level, as above. If, on the other hand, there are not any more reserve UV lights to activate when or if the energy reaches or falls below the lower limit, then, as shown at 310, the method 300 of at least one embodiment will signal (e.g., audibly or visually) that the system needs or requires maintenance.

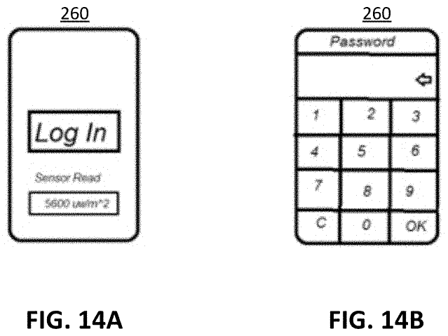

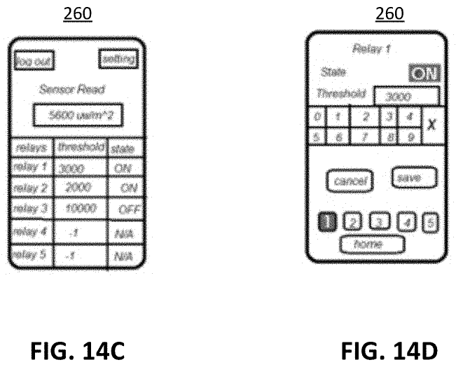

For example, there may be an interface 240, as shown in FIGS. 14A, 14B, 14C and 14D, which can be communicatively connected to the controller 220 and/or sensor(s) 210, and which can signal to a user that maintenance of the UV system may be needed. In other cases, separate indicate lamps or light emitting diodes, LEDs, can be installed on or in the system to indicate that all of the UV lights have been activated and maintenance is required or recommended. Accordingly, the degraded UV lights can then be replaced with new or replacement UV lights and the method 300 can repeat itself.

Moreover, with reference to FIGS. 14A-D, an exemplary interface 260 is shown. FIGS. 14A and 14B show an exemplary login and password screen. As shown in FIG. 14C, for example, a user may set or define the lower level or lower limit value for the UV or UV-C energy in the chamber of system 200 as measured by the sensor(s) 210, which may be uW/cm² or mW/cm². The low value or lower limit would typically be the minimum amount of energy within the irradiation chamber 40 or system 200 that would provide a desired level of disinfection of the air passing there through. For instance, depending on the type of pathogen desired to be killed or disinfected, the lower level or limit may vary from one application or implementation to another. For instance, each bacteria and virus has its own level of energy required to kill it or make it inactive. That level can also vary depending on the desired kill or inactivation percentage. UV energy at the treatment point is a key variable in the equation.

In some implementations, the controller will automatically activate a previously inactive UV light source when the energy level drops to or below the defined lower limit or threshold. In this manner, the system and method of at least one embodiment allows for monitoring the energy level of the system and maintaining a minimum predetermined level of irradiance energy without human intervention.

With reference to FIG. 14C, the interface 260, for example, can display the current measured energy level or irradiation level of the system 200 as measured by the one or more sensors 210. Additional information that can be displayed and provided by the system or interface 260 can include, for example, identification of the specific UV lights that are active or operating, an identification of the specific UV lights that have reached or passed the minimum threshold or lower limit referenced above, air flow rate (e.g., if an airflow rate sensor is included), a calculation of Fluence value (e.g., “Dosing” level) measured as uJ/cm² or mJ/cm²), etc. The calculation of Fluence/Dosing is based upon the irradiation energy at a specific treatment distance and the duration of the treatment.

For instance, duration of the pathogen exposure, in seconds, would be calculated from the volume of the chamber (V), for example in cubic-feet (which can be entered or programmed into the interface by the user), the air flow rate (R), for example in cubic-feet per second, and the resultant time (T), for example in seconds required for an sir segment to pass through the chamber. If no air flow meter is installed, then the cumulative flow rate of the fans on the inlet side of the assembly would be used. Fluence/Dosing (D) is then calculated from the UVC Irradiance (I) and the exposure duration (T). An exemplary formula is as follows: V/R=T and T×I=D If a one-foot diameter by two-foot long irradiation chamber is used, then the volume equals 1.57 cubic-feet. If Irradiance is 4.4 mW/cm² and fan speed if about 80 cubic-feet per minute, which equals 1.33 cubic inches per second, then 1.57/1.33×4.4=5.2 mJ/cm² Fluence/Dosing of the pathogens. The Fluence/Dosing of at least one embodiment of the present invention may be easily increased if required to kill or make UV resistant pathogens inactive. Increasing the length of the irradiation chamber will increase time (T) and the addition of active UV light source(s) will increase irradiance (I).

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention. This written description provides an illustrative explanation and/or account of the present invention. It may be possible to deliver equivalent benefits using variations of the specific embodiments, without departing from the inventive concept. This description and these drawings, therefore, are to be regarded as illustrative and not restrictive.

Now that the invention has been described,

What is claimed is:

1. A germicidal duct assembly, comprising:
an inlet assembly comprising at least one inlet duct,
an irradiation chamber, and
an outlet assembly comprising at least one outlet duct,
said irradiation chamber comprising a first end connected to said inlet assembly and a second end connected to said outlet assembly, wherein air flows from said inlet assembly, through said irradiation chamber and out of said outlet assembly,
wherein said irradiation chamber comprises at least one germicidal light source disposed within an interior portion thereof and along a path of the air as the air flows through said irradiation chamber to expose the air to said at least one disinfecting light source.

2. The germicidal duct assembly as recited in claim 1 further comprising a plurality of germicidal light sources with an elongated configuration.

3. The germicidal duct assembly as recited in claim 2 wherein each of said plurality of germicidal light sources extend at least substantially along a length of said irradiation chamber.

4. The germicidal duct assembly as recited in claim 2 wherein at least one of said plurality of germicidal light sources is positioned in an oblique manner relative at least another one of said plurality of germicidal light sources.

5. The germicidal duct assembly as recited in claim 2 wherein at least one of said plurality of germicidal light sources is positioned in an oblique manner relative to a longitudinal axis of said irradiation chamber.

6. The germicidal duct assembly as recited in claim 5 wherein at least two of said plurality of germicidal light sources are positioned in an oblique manner relative to a longitudinal axis of said irradiation chamber.

7. The germicidal duct assembly as recited in claim 6 wherein said at least two of said plurality of germicidal light sources extend through said longitudinal axis of said irradiation chamber.

8. The germicidal duct assembly as recited in claim 6 wherein said inlet duct comprises a light reflecting material disposed on at least a portion of an interior surface thereof, said light reflecting material being structured to reflect light from said at least two of said plurality of germicidal light sources through at least a portion of said inlet duct.

9. The germicidal duct assembly as recited in claim 1 further comprising a plurality of germicidal light sources disposed within said irradiation chamber, at least one ultraviolet light sensor, and a controller, said ultraviolet light sensor being structured to measure ultraviolet energy levels.

10. The germicidal duct assembly as recited in claim 9 wherein said plurality of germicidal light sources comprises at least one active germicidal light source and at least one back-up germicidal light source, wherein the at least one back-up germicidal light source is defined as being at least initially turned off such that no light is emitted therefrom.

11. The germicidal duct assembly as recited in claim 10 wherein, when said sensor detects an amount of energy below a predetermined level, said controller is structured to activate said back-up germicidal light source.

12. The germicidal duct assembly as recited in claim 1 further comprising at least one fan structured to facilitate a flow of air through at least a portion of said germicidal duct assembly.

13. The germicidal duct assembly as recited in claim 12 wherein said inlet assembly comprises at least one inlet vent connected to an inlet end of said at least one inlet duct.

14. The germicidal duct assembly as recited in claim 13 wherein said at least one fan comprises an inlet fan mounted to said at least on inlet vent.

15. The germicidal duct assembly as recited in claim 13 wherein said outlet assembly comprises at least one outlet vent connected to an outlet end of said at least one outlet duct.

16. The germicidal duct assembly as recited in claim 15 wherein said at least one fan comprises at least one outlet fan structured to facilitate a flow of air through at least a portion of said germicidal duct assembly.

17. The germicidal duct assembly as recited in claim 16 wherein said outlet fan is mounted proximate to said second end of said irradiation chamber.

18. A germicidal duct assembly, comprising:
an inlet assembly,
an irradiation chamber, and
an outlet assembly,
said inlet assembly comprising at least one inlet duct and at least one inlet vent,
said outlet assembly comprising at least one outlet duct and at least one outlet vent,
said irradiation chamber comprising a first end and a second end defining a longitudinal axis there between, wherein said first end of said irradiation chamber is connected to said inlet assembly and said second end of said irradiation chamber is connected to said outlet assembly, wherein air flows from said inlet assembly, through said irradiation chamber and out of said outlet assembly,
wherein said irradiation chamber comprises at least one ultraviolet light source disposed within an interior portion thereof, said at least one ultraviolet light source comprising an elongated configuration and being disposed in an oblique manner relative to a longitudinal axis of said irradiation chamber.

19. The germicidal duct assembly as recited in claim 18 further comprising a plurality of ultraviolet light sources disposed in an oblique manner relative to said longitudinal axis of said irradiation chamber.

20. The germicidal duct assembly as recited in claim 19 wherein said plurality of ultraviolet light sources extend through said longitudinal axis of said irradiation chamber.
